(19) United States
(12) Patent Application Publication
(10) Pub. No.: US 2025/0152301 A1
(43) Pub. Date: May 15, 2025

(54) Support for the Handling of Medical Instruments

(71) Applicant: KARL STORZ SE & Co KG, Tuttlingen (DE)

(72) Inventor: Klaus M. Irion, Liptingen (DE)

(73) Assignee: KARL STORZ SE & Co KG, Tuttlingen (DE)

(21) Appl. No.: 18/944,477

(22) Filed: Nov. 12, 2024

(30) Foreign Application Priority Data
Nov. 13, 2023 (DE) 102023131407.6

(51) Int. Cl. A61B 90/90; A61B 50/30; G06T 7/00

(52) U.S. Cl. CPC A61B 90/90; A61B 50/30; G06T 7/00

(57) ABSTRACT

A system for assisting in the handling of medical instruments (10) comprises a camera (42, 52, 62) for optically capturing an image of one or more medical instruments (10) and for providing an image signal representing the captured image, an image evaluation device (70) for receiving the image signal, for identifying one or more medical instruments (10) depicted in the image represented by the image signal and for providing identification data identifying the depicted instruments (10), and a data transmission device (56, 66) for sending identification data to a data receiving device (88) of a data device (22) permanently connected to the autoclavable instrument carrier (20).

This application claims priority to German Patent Application No. 102023131407.6 filed Nov. 13, 2023, and entitled “Unterstützung der Handhabung medizinischer Instrumente,” which is incorporated herein by reference.

The present invention relates to a system, to an autoclavable instrument carrier, to an autoclavable sterile container and to a method for assisting in the handling of medical instruments.

WO 2009/003231 A1 (Mems-ID Pty Ltd.) describes a system for identifying surgical instruments. The surgical instruments are provided with RFID tags that can withstand sterilization. A removable scanning device is provided on a container, which can be a tray, for holding the surgical instruments, by means of which scanning device the RFID tags on surgical instruments in the container are scanned. The scanning device is removed from the container before sterilization and then reattached to the container.

WO 2009/076452 A2 (Robotic Systems & Technologies, Inc.; also published as US 2011/0005342 A1, US 2011/0262250 A1, U.S. Pat. No. 7,997,847 B2, U.S. Pat. No. 8,567,880 B2) describes a method for handling a plurality of surgical instruments for cleaning. An insert having a predetermined configuration for receiving at least one type of surgical instrument is identified. Each type of the plurality of surgical instruments is identified using an optical device or an RFID reader. Each of the identified surgical instruments is oriented using an automated device. Each type of surgical instrument is positioned in one or more regions of the insert using the automated device.

US 2016/0085922 A1 (Spinal Generations, LLC; also published as U.S. Pat. No. 10,552,574 B2) describes the identification of a medical device. The medical device is identified optically using a camera and image recognition and/or using an RFID reader that reads an RFID tag on the medical device. The RFID reader is in particular located in an operating room. A tray has an RFID tag that contains information about the contents of the tray.

DE 10 2015 108 264 A1 (Aesculap AG; also published as WO 2016/188959 A1, EP 3 302 340 B1, US 2018/0153639 A1, U.S. Pat. No. 10,368,958 B2, CN 107708599 A) describes a surgical container content detection system. A carrier in the form of a mat is arranged in a sterilization container. The carrier comprises a carrier module with a transponder reader, in particular an RFID reader, a storage device for temporarily storing recorded data, an energy storage for the self-supply of the carrier module and a wireless data transmission device. The RFID reader detects identification elements on objects in the sterilization container. Information about the object identified by the detected identification element is transmitted wirelessly to outside the sterilization container by the wireless data transmission device.

WO 2016/023097 A1 (Synaptive Medical, Inc.; also published as CA 2 957 794 A1, CA 2 957 794 C, US 2017/0243157 A1, U.S. Pat. No. 10,592,857 B2) describes a system and a method for managing equipment for a medical application. An input device for identifying equipment includes a tracking camera, an optical camera, an RFID receiver, a structured light camera, or a stereo camera pair.

One object of the present invention is to provide an improved system, an improved autoclavable instrument carrier, an improved autoclavable sterile container and an improved method for assisting in the handling of medical instruments.

This object is achieved by the subject matters of the independent claims.

Further embodiments are defined in the dependent claims.

A system for assisting in the handling of medical instruments comprises a camera for optically capturing an image of one or more medical instruments and for providing an image signal representing the captured image, an image evaluation device for receiving the image signal, for identifying one or more medical instruments depicted in the image represented by the image signal and for providing identification data identifying the depicted instruments, and a data transmission device for sending identification data to a data receiving device of a data device permanently connected to an autoclavable instrument carrier.

The system is, in particular, provided and configured to simplify the handling of autoclavable medical instruments immediately before and/or after autoclaving. Handling is, in particular, simplified by generating, recording or updating data similar to an inventory.

The camera can be provided to capture light in the infrared spectral range and/or in the spectral range visible to the healthy human eye and/or in the ultraviolet spectral range. The camera can capture a monochromatic or a multicolor image and generate a corresponding image signal. The camera can further be provided and configured for monocular imaging or for stereoscopic imaging. The camera can generate an analog or a digital image signal.

The image evaluation device can be integrated into a housing with the camera or can be configured separately from it. In particular, the image sensor of the camera can be integrated with the image evaluation device on a semiconductor component (die) or in an electronic component. Alternatively, the image evaluation device can be configured as a computer with suitable software. The analog or digital image signal can be transmitted to the image evaluation device electrically, optically or in another way.

The image evaluation device is, in particular, provided and configured to identify a medical instrument based on optically recognizable features, such as size, shape and/or colors. For this purpose, the image evaluation device comprises in particular a database in which corresponding features are stored and assigned to individual types of medical instruments or to individual medical instruments.

Alternatively or additionally, the image evaluation device can be provided and configured to detect a code on a medical instrument, for example a barcode, a QR code or another one- or two-dimensional monochromatic or polychromatic code in the form of stripes, dots, rectangles or alphanumeric characters. Such an individual code can enable or simplify the differentiation between medical instruments of the same construction and thus also the recording of an exact history for each individual medical instrument. Accordingly, the identification data generated by the image evaluation device include either only the type or design of the medical instrument or, for example, also a batch number or a serial number of the medical instrument.

The data transmission device comprises in particular a transmitter for sending the identification data or other data to the data receiving device of the data device permanently connected to the autoclavable instrument carrier. The transmitter can be provided and configured to send the data in an analog or digital format. The transmitter can be provided and configured for predominantly inductive or predominantly capacitive or for electromagnetic data transmission.

The data transmission device can further be provided and configured to receive or read identification data or other data from a data receiving device permanently connected to an instrument carrier.

The data transmission device complies, for example, with the near field communication (NFC) standard or another radio-frequency identification (RFID) standard.

In particular, the data device is permanently mechanically connected to the instrument carrier in that it is not separated from the instrument carrier by the statically or dynamically generated forces occurring during the intended use of the instrument carrier. For example, the data device is mechanically connected to the instrument carrier by a snap-in or clamp connection.

The data device can further be permanently mechanically connected to the instrument carrier in that it cannot be separated from the instrument carrier without the use of tools and without being destroyed. For example, the data device is mechanically connected to the instrument carrier by a screw or rivet connection.

Many medical instruments are too small to accommodate an RFID tag, for example. Also, this does not make economic sense for many inexpensive medical instruments. Furthermore, reading many RFID tags in a small space is difficult. Reading is further complicated by the usually metallic structure of the instrument carrier, which acts as a Faraday cage and shields at least electrical and electromagnetic fields very well.

Automated optical identification and joint storage of identification data in a memory permanently connected to the instrument carrier can solve these problems. The individual medical instrument does not need to have an RFID tag, and there is no need to read a large number of RFID tags in a small space. Nevertheless, the memory permanently connected to the instrument carrier can be written to and read without contact.

A system as described herein further comprises, in particular, an autoclavable instrument carrier for storing, transporting and keeping medical instruments ready, and an autoclavable data device permanently connected to the autoclavable instrument carrier and having a data receiving device for receiving data and a repeatedly writable and readable memory for storing data received by the data receiving device.

The instrument carrier is configured in particular as a tray with a rectangular flat region and a surrounding edge. Aside from the autoclavable data device, the instrument carrier can correspond to a standard in order to be interchangeable with conventional instrument carriers, to replace them and to be autoclaved together with them or instead of them in conventional autoclaves. The instrument carrier is made in particular from a metal mesh or a perforated metal sheet and is therefore also referred to as a sieve or instrument sieve. The instrument carrier can be provided and configured to accommodate one or more medical instruments at the same time.

The data device is in particular an RFID tag. The data receiving device corresponds in particular to an RFID standard for predominantly capacitive, predominantly inductive or predominantly electromagnetic communication. Alternatively or additionally, the data receiving device can be provided and configured for optically receiving data. The data receiving device is also provided and configured in particular for transmitting data stored in the memory of the data device. The data device is, in particular, configured to read data from the memory of the data device upon receiving a request signal and to send it to the sender of the request signal.

The memory is configured in particular as a non-volatile memory that stores data even without a power supply. In that case, the data device does not need to have an energy storage to continuously supply power to the memory.

The data device permanently connected to the instrument carrier enables identity data of medical instruments held by the instrument carrier to be stored directly and permanently on the instrument carrier. The memory of the data device can be read at any time with little technical effort and only locally available infrastructure—for example an RFID reader—in order to obtain the identity data of the medical instruments in or on the instrument carrier. A data connection to another database—for example on a hospital server—is not required.

A system as described herein further comprises, in particular, an autoclavable energy receiving device permanently connected to the autoclavable instrument carrier for receiving power in the form of light and for providing electrical power for the data device (22).

A system as described herein further comprises, in particular, a light source for providing power in the form of light for the power receiving device permanently connected to the autoclavable instrument carrier.

The light source is in particular a monochromatic or narrow-spectrum-emitting and directed light source, for example a laser. In particular, the light source is arranged and aligned such that it irradiates or substantially irradiates the power receiving device only when the autoclavable instrument carrier is arranged and oriented in the intended manner. The system may include a controller that activates the light source only when the data device requires electrical power, in particular when receiving, writing to memory, reading or sending data. The system may include a controller that directs the light source toward the power receiving device.

The spectrum of the light source is in particular tailored to the spectral sensitivity of the power receiving device so that both spectra have as large an overlap as possible. Power can be transmitted by light in the infrared spectral range and/or in the spectral range visible to the healthy human eye and/or in the ultraviolet spectral range.

The power receiving device is integrated in particular into the data device and forms an autoclavable unit therewith.

Due to the power supply by the light source and the power receiving device, no energy storage is required in the data device. This can allow for miniaturization and lower manufacturing costs as well as higher temperature resistance of the data device.

A system as described herein further comprises, in particular, a projection device for visibly projecting an intended target position of a medical instrument onto a surface of the autoclavable instrument carrier.

The projection device is, in particular, provided and configured to project an outline of a medical instrument onto the instrument carrier in the arrangement and orientation intended for the medical instrument. For example, a laser beam can describe the outline of the medical instrument.

The projection of the intended position and optionally also of the orientation of the medical instrument onto the instrument carrier can simplify the manual equipment of the instrument carrier and make it more reliable.

A system as described herein further comprises, in particular, a database in which intended positions of medical instruments on a surface of the autoclavable instrument carrier are stored.

The database contains, in particular for different medical procedures, different equipment of the autoclavable instrument carriers, each adapted to the specific medical procedure. Predetermined locations and orientations for all contained medical instruments can be stored for each equipment.

An autoclavable instrument carrier for storing, transporting and keeping medical instruments ready comprises an autoclavable data device permanently connected to the autoclavable instrument carrier with a repeatedly writable and readable memory and a data transmission device for receiving and sending data and an autoclavable energy receiving device permanently connected to the autoclavable instrument carrier for receiving power in the form of light and for providing electrical power for the data device.

The autoclavable instrument carrier is, in particular, provided to be part of a system as described herein to assist in the handling of medical instruments. The autoclavable instrument carrier has in particular features, properties and functions as described herein in connection with the autoclavable instrument carrier of the described system.

The instrument carrier is configured in particular as a tray with a rectangular flat region and a surrounding edge. Aside from the autoclavable data device, the instrument carrier can correspond to a standard in order to be interchangeable with conventional instrument carriers, to replace them and to be autoclaved together with them or instead of them in conventional autoclaves. The instrument carrier is made in particular from a metal mesh or a perforated metal sheet and is therefore also referred to as a sieve or instrument sieve. The instrument carrier can be provided and configured to accommodate one or more medical instruments at the same time.

The data device is in particular an RFID tag. The data receiving device corresponds in particular to an RFID standard for predominantly capacitive, predominantly inductive or predominantly electromagnetic communication. Alternatively or additionally, the data receiving device can be provided and configured for optically receiving data. The data receiving device is also provided and configured in particular for transmitting data stored in the memory of the data device. The data device is, in particular, configured to read data from the memory of the data device upon receiving a request signal and to send it to the sender of the request signal.

In particular, the data device is permanently mechanically connected to the instrument carrier in that it is not separated from the instrument carrier by the statically or dynamically generated forces occurring during the intended use of the instrument carrier. For example, the data device is mechanically connected to the instrument carrier by a snap-in or clamp connection.

The data device can further be permanently mechanically connected to the instrument carrier in that it cannot be separated from the instrument carrier without the use of tools and without being destroyed. For example, the data device is mechanically connected to the instrument carrier by a screw or rivet connection.

The memory is configured in particular as a non-volatile memory that stores data even without a power supply. In that case, the data device does not need to have an energy storage to continuously supply power to the memory.

The power receiving device can be provided and configured to receive power in the form of light in the infrared spectral range, in the spectral range visible to the healthy human eye and/or in the ultraviolet spectral range. The power receiving device is integrated in particular into the data device and forms an autoclavable unit therewith.

The data device permanently connected to the instrument carrier enables identity data of medical instruments held by the instrument carrier to be stored directly and permanently on the instrument carrier. The memory of the data device can be read at any time with little technical effort and only locally available infrastructure—for example an RFID reader—in order to obtain the identity data of the medical instruments in or on the instrument carrier. A data connection to another database—for example on a hospital server—is not required.

Due to the power supply by the light source and the power receiving device, no energy storage is required in the data device. This can allow for miniaturization and lower manufacturing costs as well as higher temperature resistance of the data device.

Alternatively or additionally, the identity data of the medical instruments can be transmitted to a separate database via a data connection and stored there and/or read from a separate database. Storing identity data both in the data device on the autoclavable instrument carrier and in a separate database can significantly reduce the risk of losing identity data. The integrity of the identity data can be confirmed by comparing identity data stored in the data device on the autoclavable instrument carrier and identity data stored in a separate database. Even if, temporarily, identity data cannot be read from the data device on the autoclavable instrument carrier or if they are permanently lost, they can most likely be read from the separate database, and vice versa.

An autoclavable instrument carrier as described herein further comprises, in particular, a barcode or other one- or two-dimensional optically readable code or other optically readable device for identifying the autoclavable instrument carrier.

An autoclavable instrument carrier for storing, transporting and keeping medical instruments ready comprises an autoclavable data device permanently connected to the autoclavable instrument carrier and with a repeatedly writable and readable memory and a data transmission device for sending and receiving data and a barcode or other one- or two-dimensional optically readable code or other optically readable device for identifying the autoclavable instrument carrier.

The barcode or other one- or two-dimensional optically readable code or other optically readable device attached to the autoclavable instrument carrier provides an additional means of identifying the autoclavable instrument carrier. If the identity of the autoclavable instrument carrier is known, the contents or equipment of the autoclavable instrument carrier can be read from a database. Furthermore, the optically readable code or the optically readable device can enable verification to rule out the possibility that, for example, a data device on another instrument carrier has been erroneously read by means of an RFID reader.

An autoclavable sterile container for storing, transporting and keeping ready one or more instrument carriers, each with one or more medical instruments, comprises an autoclavable data device permanently connected to the autoclavable sterile container and having a repeatedly writable and readable memory and a data transmission device for sending and receiving data, and an autoclavable energy receiving device permanently connected to the autoclavable sterile container for receiving power in the form of light and for providing electrical power for the data device.

The autoclavable sterile container is, in particular, provided and configured to accommodate one or more autoclavable instrument carriers as described herein. The autoclavable sterile container is, in particular, provided and configured for use with a system as described herein.

The autoclavable sterile container has in particular a lid or other device for closing. When closed, the autoclavable sterile container prevents in particular germs or other particles and optionally also liquids or gases from entering the autoclavable sterile container. In this way, sterile goods contained in the autoclavable sterile container remain sterile until the sterile container is opened again.

The autoclavable sterile container can comprise a membrane that allows the penetration of hot steam to sterilize the medical instruments on the instrument carriers, the instrument carriers and the inner surfaces of the sterile container during the autoclaving process, but prevents germs or other particles from entering. The membrane can be provided on a lid or other closing device or can form the lid or other closing device in whole or in part.

Furthermore, the autoclavable sterile container can have similar features, properties and functions to an autoclavable instrument carrier as described herein.

In particular, the data device is arranged entirely or partially on an outside of the sterile container so that it can be written to or read from outside. The data device is in particular an RFID tag. The data receiving device corresponds in particular to an RFID standard for predominantly capacitive, predominantly inductive or predominantly electromagnetic communication. Alternatively or additionally, the data receiving device can be provided and configured for optically receiving data. The data receiving device is also provided and configured in particular for transmitting data stored in the memory of the data device. The data device is, in particular, configured to read data from the memory of the data device upon receiving a request signal and to send it to the sender of the request signal.

In particular, the data device is permanently mechanically connected to the sterile container in that it is not separated from the sterile container by the statically or dynamically generated forces occurring during the intended use of the sterile container. For example, the data device is mechanically connected to the sterile container by a snap-in or clamp connection.

The data device can further be permanently mechanically connected to the sterile container in that it cannot be separated from the sterile container without the use of tools and without being destroyed. For example, the data device is mechanically connected to the sterile container by a screw or rivet connection.

The memory is configured in particular as a non-volatile memory that stores data even without a power supply. In that case, the data device does not need to have an energy storage to continuously supply power to the memory.

The power receiving device can be provided and configured to receive power in the form of light in the infrared spectral range, in the spectral range visible to the healthy human eye and/or in the ultraviolet spectral range. The power receiving device is integrated in particular into the data device and forms an autoclavable unit therewith.

The data device permanently connected to the sterile container enables identity data of medical instruments accommodated in the sterile container to be stored directly and permanently on the sterile container. The memory of the data device can be read at any time with little technical effort and only locally available infrastructure—for example an RFID reader—in order to obtain the identity data of the medical instruments in the sterile container. A data connection to another database—for example on a hospital server—is not required.

Due to the power supply by the light source and the power receiving device, no energy storage is required in the data device. This can allow for miniaturization and lower manufacturing costs as well as higher temperature resistance of the data device.

An autoclavable sterile container as described herein further comprises, in particular, a barcode or other one- or two-dimensional optically readable code or other optically readable device for identifying the autoclavable sterile container.

An autoclavable sterile container for storing, transporting and keeping ready one or more instrument carriers, each with one or more medical instruments, comprises an autoclavable data device permanently connected to the autoclavable sterile container and having a repeatedly writable and readable memory and a data transmission device for sending and receiving data and a barcode or another one- or two-dimensional optically readable code or another optically readable device for identifying the autoclavable sterile container.

The barcode or other one- or two-dimensional optically readable code or other optically readable device attached to the autoclavable instrument carrier provides an additional means of identifying the autoclavable instrument carrier. If the identity of the autoclavable instrument carrier is known, the contents or equipment of the autoclavable instrument carrier can be read from a database. Furthermore, the optically readable code or the optically readable device can enable verification to rule out the possibility that, for example, a data device on another instrument carrier has been erroneously read by means of an RFID reader.

In case of an autoclavable instrument carrier as described herein or an autoclavable sterile container as described herein, the energy receiving device comprises in particular a photovoltaic cell or an optoelectric converter based on gallium arsenide.

A method for assisting in the handling of medical instruments comprises optically capturing an image of one or more medical instruments, generating an image signal representing the captured image, transmitting the image signal to an image evaluation device, having the image signal evaluated by the image evaluation device to identify the medical instrument or instruments depicted in the image, generating identity data representing the identities of the identified medical instrument or instruments, transmitting identity data and storing the identity data in a memory permanently connected to an instrument carrier or comparing the identity data with identity data stored in the memory permanently connected to the instrument carrier.

The method can be carried out in particular with a system as described herein, with an autoclavable instrument carrier as described herein and/or with an autoclavable sterile container as described herein.

In particular, the method can simplify the handling of autoclavable medical instruments immediately before and/or after autoclaving. Handling is, in particular, simplified by generating, recording or updating data similar to an inventory.

The image can be in the infrared spectral range and/or in the spectral range visible to the healthy human eye and/or in the ultraviolet spectral range. The image captured can be a monochromatic or multicolor image and can generate a corresponding image signal. The image can be captured monocularly or stereoscopically provided and configured. The image signal can be analog or digital.

The medical instrument will be identified based on visually recognizable features, such as size, shape and/or colors. For this purpose, the image evaluation device compares features in the image represented by the image signal in particular with corresponding features that are stored in a database and assigned to individual types of medical instruments or single individual medical instruments.

Alternatively or additionally, a code on a medical instrument can be detected, for example a barcode, a QR code or another one- or two-dimensional monochromatic or polychromatic code in the form of stripes, dots, rectangles or alphanumeric characters. Such an individual code can enable or simplify the differentiation between medical instruments of the same construction and thus also the recording of an exact history for each individual medical instrument. Accordingly, the identification data generated include either only the type or design of the medical instrument or, for example, also a batch number or a serial number of the medical instrument.

The transmission of identity data takes place in particular in accordance with the near field communication (NFC) standard or another radio-frequency identification (RFID) standard.

In particular, the generated identity data are transferred to the memory permanently connected to the instrument carrier and stored therein. Alternatively, identity data stored in the memory permanently connected to the instrument carrier are read from the latter, transmitted to a device for comparison and compared there with the generated identity data.

Many medical instruments are too small to accommodate an RFID tag, for example. Also, this does not make economic sense for many inexpensive medical instruments. Furthermore, reading many RFID tags in a small space is difficult. Reading is further complicated by the usually metallic structure of the instrument carrier and other medical instruments therein, which act as a Faraday cage and shield at least electrical and electromagnetic fields very well.

Automated optical identification and joint storage of identification data in a memory permanently connected to the instrument carrier or the reading of identification data from it for subsequent comparison can solve these problems. The individual medical instrument does not need to have an RFID tag, and there is no need to read a large number of RFID tags in a small space. Nevertheless, the memory permanently connected to the instrument carrier can be written to and read without contact.

In one method as described herein, having the image signal evaluated by the image evaluation device comprises in particular capturing the arrangement of the medical instruments depicted in the image, wherein the captured arrangement is stored as the target arrangement of the medical instruments (10).

In particular, the arrangement of one or more medical instruments on an instrument carrier is captured, wherein the arrangement optionally comprises the orientation of each medical instrument. Storing the captured arrangement, optionally comprising the captured orientation, as a target arrangement can be part of a system learning phase or training phase. During said learning phase or training phase, the target arrangement, optionally comprising the target orientation, is generated in particular manually, then recorded as a reference for each subsequent equipment of an instrument carrier and later used as a reference.

The target arrangement can be stored in a memory permanently connected to the instrument carrier or in a database at another location.

A method as described herein further comprises, in particular, determining a target arrangement of an identified medical instrument on an instrument carrier and projecting the medical instrument in the target arrangement onto the instrument carrier.

Determining the target arrangement, optionally comprising a target orientation, comprises in particular selecting one of several stored target arrangements, namely the target arrangement that is assigned to the identified medical instrument. Determining the target arrangement further includes reading the selected target arrangement. The target arrangement can be read from a memory that is permanently mechanically connected to the instrument carrier or, for example, from a separate database.

Projecting the medical instrument in the target arrangement comprises in particular projecting the outline of the medical instrument in the target arrangement onto the instrument carrier. For example, the outline of the medical instrument is quickly and repeatedly scanned by a guided laser beam on the instrument carrier, so as to produce a continuous image that is motionless in the perception by the human eye. Alternatively, the outline or a detailed image of the identified medical instrument can be projected onto the instrument carrier.

A method as described herein further comprises, in particular, optically capturing a further image of the medical instrument(s) on the instrument carrier, generating a further image signal that represents the captured further image, transmitting the further image signal to the image evaluation device, having the image signal evaluated by the image evaluation device in order to determine the arrangement of the medical instrument(s) depicted in the further image, and generating a notification signal depending on whether the determined arrangement of the medical instrument(s) corresponds to their target arrangement.

The further image is captured in particular after the target arrangement of an identified medical instrument has been determined and projected onto the instrument carrier and the medical instrument has been placed on the instrument carrier. The following method steps serve to verify the correct arrangement of the medical instrument and to provide feedback to a person that equips the instrument carrier. Alternatively, the further image can be captured, for example, during automated equipment of an instrument carrier by a robotic arm in order to verify whether the medical instrument has been placed correctly, i.e., arranged and optionally also oriented according to the target arrangement.

The notification signal is, for example, an acoustic or optical signal for a person equipping the instrument carrier. Alternatively, the notification signal can be an electrically, optically or otherwise transmitted signal that triggers or allows the continuation of the equipment process or triggers a correction of the arrangement of the medical instrument.

In a method as described herein, the generation of the notification signal is in particular further dependent on whether the identity data of the identified medical instrument(s) was successful stored in the memory that is permanently connected to the instrument carrier.

A method as described herein further comprises, in particular, setting the attribute “non-sterile” to a medical instrument if the comparison of the identity data shows that the medical instrument is no longer arranged on the instrument carrier.

The comparison and setting of the attribute take place in particular after the instrument carrier has been put into use in an operating theater or at another location for a medical procedure and has been removed from a sterile container, for example.

The attribute “non-sterile” is set in particular in the memory permanently connected to the instrument carrier. Alternatively or additionally, the attribute “non-sterile” is set in another memory, for example in a central database in which all medical instruments of a medical facility are inventoried. At the same time, a counter can be incremented that counts the number of uses of the medical instrument.

A method as described herein further comprises, in particular, introducing the instrument carrier into a sterile container, reading the identity data of the identified medical instrument(s) from the memory permanently connected to the instrument carrier, transmitting the identity data of the identified medical instrument(s) to a memory permanently connected to the sterile container, and storing the identity data of the identified medical instrument(s) in the memory permanently connected to the sterile container.

Alternatively, the identity data of the identified medical instrument(s) are read from another memory, for example from a database, which stores which medical instruments are placed in which instrument carrier.

The reading of the identity data of the medical instrument(s) from the memory permanently connected to the instrument carrier is carried out in particular in accordance with an RFID standard. The transfer of the identity data of the medical instrument(s) takes place in particular in accordance with an RFID standard.

Storing the identity data in a memory permanently connected to the sterile container can make the identity data accessible from outside even if the sterile container—for example, acting as a Faraday cage—would prevent a memory on an instrument carrier inside the sterile container from being read. For this purpose, the memory permanently connected to the sterile container can be read, in particular via a transmitting and receiving device on an outside or an outer surface of the sterile container.

In the following, embodiments are explained in more detail with reference to the attached figures. In the drawings:

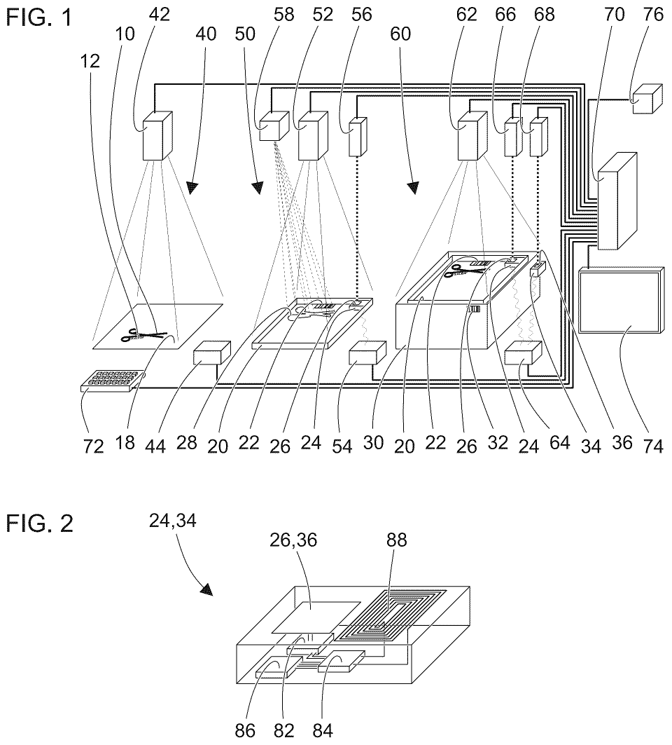

FIG. 1 shows a schematic representation of a system for assisting in the handling of medical instruments;

FIG. 2 shows a schematic representation of a data device of the system of FIG. 1;

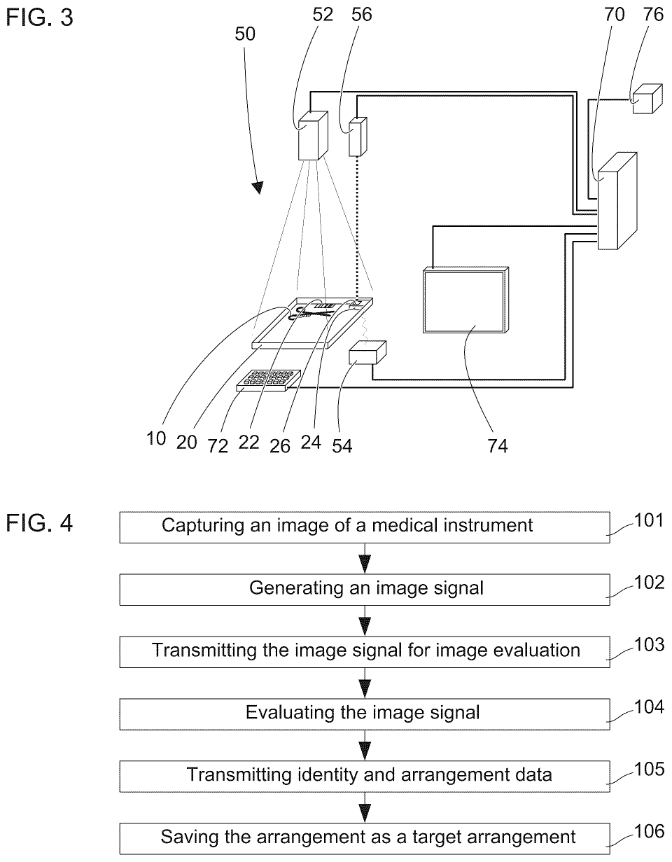

FIG. 3 shows a schematic representation of a further system for assisting in the handling of medical instruments;

FIG. 4 shows a schematic flowchart of a method for assisting in the handling of medical instruments;

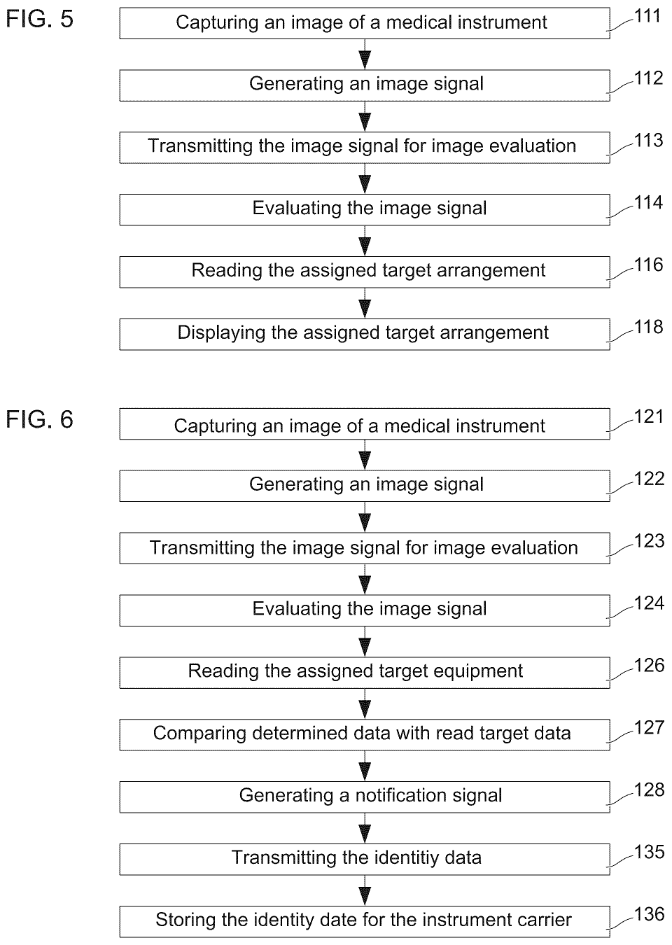

FIG. 5 shows a schematic flowchart of a method for assisting in the handling of medical instruments;

FIG. 6 shows a schematic flowchart of a method for assisting in the handling of medical instruments;

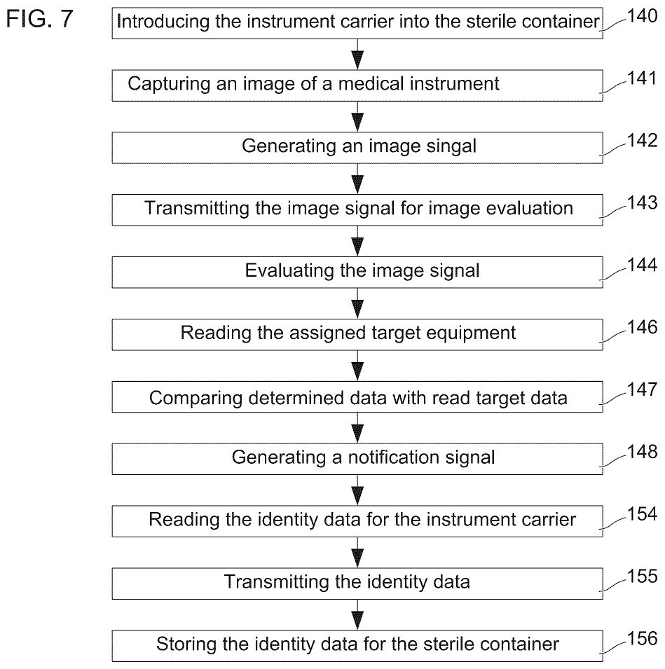

FIG. 7 shows a schematic flowchart of a method for assisting in the handling of medical instruments; and

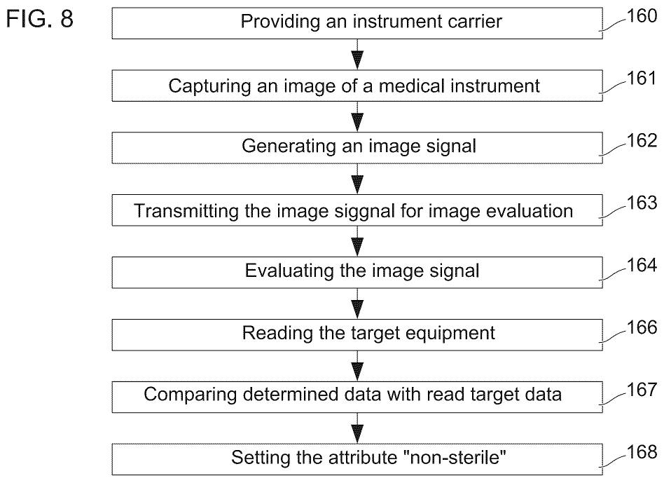

FIG. 8 shows a schematic flowchart of a method for assisting in the handling of medical instruments.

FIG. 1 shows a schematic representation of a system for assisting in the handling of medical instruments 10, in particular after cleaning, immediately before or after sterilization and before use. FIG. 1 shows a medical instrument having an optically readable code 12. The medical instrument can be placed on a storage surface 18.

An instrument carrier 20 has an optically readable code 22, for example a barcode, and an RFID tag 24. The optical code 22 and the RFID tag 24 are in particular permanently mechanically connected to the instrument carrier 20, and therefore, when a tool is not used, cannot be separated from the instrument carrier 20 without being destroyed. In the example shown, the optically readable code 22 is arranged on a straight edge section and the RFID tag 24 is arranged in a corner of the interior with an essentially rectangular basic shape. Alternatively, the optically readable code 22 and the RFID tag 24 can be arranged at other locations in or on the instrument carrier, for example can be integrated into a side wall of the instrument carrier 20.

In the example shown, the RFID tag 24 includes a photoelectric component 26 for receiving power in the form of light and for providing electrical power to electronic circuits of the RFID tag 24.

The instrument carrier 20 is made, for example, from metal mesh or a perforated metal sheet and is therefore also referred to as a sieve or instrument sieve. The instrument carrier 20 may conform to an industry standard to replace conventional instrument carriers. The instrument carrier 20 is sterilizable and in particular autoclavable, so it will not be damaged or altered even by repeated exposure to pure steam at a temperature in the range of 140 degrees Celsius.

In the situation shown, an outline 28 of the medical instrument 10 is projected onto the instrument carrier in an intended arrangement.

A sterile container 30 has an optically readable code 32, for example a barcode, and an RFID tag 34. The optical code 32 and the RFID tag 34 are in particular permanently mechanically connected to the sterile container 30, and therefore, when a tool is not used, cannot be separated from the sterile container 30 without being destroyed. In the example shown, the optically readable code 22 and the RFID tag 34 are arranged on outer surface regions of the sterile container 30. Alternatively, the optically readable code 32 and the RFID tag 34 can be arranged at other locations in or on the sterile container. For example, the RFID tag 34 can be integrated into a side wall of the sterile container 30.

In the example shown, the RFID tag 34 includes a photoelectric component 36 for receiving power in the form of light and for providing electrical power to electronic circuits of the RFID tag 34.

The sterile container 30 is provided and configured to accommodate one or more instrument carriers 20. The sterile container 30 is in particular made of gas-tight material, for example a metal sheet, and can be closed by a lid (not shown) in a gas-tight manner or at least in a manner that prevents the penetration of liquids and particles. The sterile container 30 may conform to an industry standard to replace conventional sterile containers. The sterile container 30 is sterilizable and in particular autoclavable, so it will not be damaged or altered even by repeated exposure to pure steam at a temperature in the range of 140 degrees Celsius. The sterile container 30 is provided to accommodate in the same way medical instruments 10 and instrument carriers 20 that can be sterilized, in particular during and after sterilization.

In the situation or configuration shown in FIG. 1, several instrument carriers 20 are already arranged in the sterile container 30, with the uppermost instrument carrier 20 being visible.

A first region 40 for identifying the medical instrument 10 primarily comprises the storage surface 18 on which the medical instrument is placed in the situation shown. The first region 40 and objects arranged therein are optically captured by a first camera 42. The first camera 42 captures all medical instruments 10 placed on the storage surface 18. Optionally, the first camera 42 can capture optically readable codes 12 on medical instruments 10.

Furthermore, an RFID reader 44 for reading RFID tags located in the first region 40 is provided near the storage surface 18 and the first region 40. As indicated in FIG. 1, the RFID reader 44 can be arranged next to the first region 40. Alternatively, the RFID reader 44 can be arranged, for example, below the storage surface 18. In that case, the storage surface 18 is formed by a component that is transparent to the electrical, magnetic or electromagnetic alternating fields via which the RFID reader 44 communicates with RFID tags, or does not significantly attenuate them.

In the example shown in FIG. 1, the medical instrument does not have an RFID tag.

A second region 50 is provided for equipping an instrument carrier 20. In FIG. 1, the instrument carrier 20 is arranged in the intended position and orientation in the second region 50. The intended position and orientation of the instrument carrier 20 can be mechanically defined by stops.

The second region 50 and objects arranged therein are optically captured by a second camera 52. The second camera 52 captures, in particular, the instrument carrier 20 and medical instruments 10 placed on the instrument carrier 20. Optionally, the second camera 52 can capture optically readable codes 12 on medical instruments and the optically readable code 22 on the instrument carrier 20.

Furthermore, an RFID writer 54 is provided near the second region 50 for writing data into RFID tags located in the second region 50. The RFID writer 54 can at the same time be an RFID reader for reading data from RFID tags. As indicated in FIG. 1, the RFID writer 54 can be arranged next to the second region 50. Alternatively, the RFID writer 54 can be arranged below the intended position of the instrument carrier 20. In that case, the storage surface on which the instrument carrier 20 is to be arranged is formed by a component that is transparent to the electrical, magnetic or electromagnetic alternating fields via which the RFID writer 54 communicates with RFID tags, or does not significantly attenuate them.

A first light source 56 generates light that can be converted into electrical power by the photoelectric component 26 of the RFID tag 24 on the instrument carrier 20. For this purpose, the emission spectrum of the first light source 56 is, in particular, matched to the absorption spectrum of the photoelectric component 26, so that as large a part as possible of the electrical power supplied to the first light source 56 is converted back into electrical power by the photoelectric component 26.

The first light source 56 generates in particular only a narrow light beam or light cone and primarily illuminates the region in which the photoelectric component 26 is arranged in the intended arrangement of the instrument carrier 20. The first light source 56 comprises in particular one or more lasers or light-emitting diodes.

In the intended arrangement, a projection device 58 projects the outline 28 of the medical instrument 10 onto the instrument carrier 20. The projection device 58 comprises in particular a laser and a device for controllably directing the laser beam so that it describes the outline 28. Alternatively, the projection device 58 may comprise another light source and, for example, a controllable micromirror array, a controllable liquid crystal matrix or a slide.

A third region 60 is provided for equipping a sterile container 30 with one or more instrument carriers 20. In FIG. 1, the sterile container 30 is arranged in the intended position and orientation in the third region 60. The intended position and orientation of the sterile container 30 can be mechanically defined by stops.

The third region 60 and objects arranged therein are optically captured by a third camera 62. The third camera 62 captures in particular the sterile container 30, the instrument carrier 20 placed therein (in the case of a stack of instrument carriers: the topmost instrument carrier 20) and medical instruments 10 placed on the instrument carrier 20. Optionally, the third camera 62 can capture optically readable codes 12 on medical instruments and the optically readable code 22 on the instrument carrier 20.

Optionally, a further device (not shown in FIG. 1) for capturing the optically readable code 32 can be provided on the sterile container 30.

Furthermore, an RFID reader and writer 64 is provided near the third region 60 for reading data from RFID tags and for writing data into RFID tags located in the third region 60. As indicated in FIG. 1, the RFID reader and writer 64 can be arranged next to the second region 50. Alternatively, the RFID reader and writer 64 can be arranged below the intended position of the sterile container 30. In that case, the storage surface on which the sterile container 30 is to be arranged is formed by a component that is transparent to the electrical, magnetic or electromagnetic alternating fields via which the RFID writer 54 communicates with RFID tags, or does not significantly attenuate them.

A second light source 66 generates light that can be converted into electrical power by the photoelectric component 26 of the RFID tag 24 on the instrument carrier 20. The second light source 66 resembles in terms of its features, properties and functions in particular the first light source 56 for the photoelectric component 26 on the instrument carrier 20 in the second region.

A third light source 68 generates light that can be converted into electrical power by the photoelectric component 36 of the RFID tag 34 on the sterile container 30. The third light source 68 resembles in terms of its features, properties and functions in particular the first light source 56 and the second light source 66. The spectrum of the third light source 68 and the region illuminated by the third light source 68 are matched to the spectral properties and the intended arrangement of the photoelectric component 36 on the RFID tag 34 on the sterile container 30.

A computer 70 is connected to the first camera 42, the RFID reader 44, the second camera 52, the RFID writer 54, the projection device 58, the third camera 62, the RFID reader and writer 64 via electrical or optical data lines indicated in FIG. 1. As indicated in FIG. 1, the computer 70 can be optionally connected to the first light source 56, the second light source 66 and the third light source 68 by further electrical or optical lines.

The computer 70 is further connected to an input device 72 (shown as a keyboard in FIG. 1), an output device 74 (shown as a screen or display in FIG. 2) and a database 76 via electrical or optical data lines indicated in FIG. 1. The input device 72 and the output device 74 together form a user interface.

Point-to-point connections are indicated in FIG. 1. Alternatively, another topology is possible, for example a bus structure, a ring-shaped or a star-shaped topology. Furthermore, a radio connection can be provided as an alternative to each individual wired communication channel, for example in accordance with the WLAN, Bluetooth, ZigBee, etc standards.

Deviating from the representation in FIG. 1, several devices can be integrated in one housing. For example, the database 76, the input device 72 and/or the output device can be integrated into the computer 70.

The computer 70 is provided and configured to evaluate an image signal of the first camera 42, to identify a medical instrument 10 depicted in the image represented by the image signal, to generate corresponding identity data and to determine a target arrangement of the identified instrument 10 on the instrument carrier 20. The computer 70 is further provided and configured to control the projection device 58 such that it projects an outline 28 of the identified instrument 10 in the determined target arrangement onto the instrument carrier 20 or displays the target arrangement in another way, for example on the output device 74.

The computer 70 is further provided and configured to receive an image signal representing an image optically captured by the second camera 52, to identify medical instruments 10 depicted in the image, to generate corresponding identity data, to determine the arrangement of the identified medical instruments 10 relative to the instrument carrier 20, to compare the identities of the identified medical instruments with a target equipment, to compare the determined arrangement of the identified medical instruments 10 with a target arrangement and to generate a notification signal depending on these comparisons.

The computer 70 is further provided and configured to receive an image signal representing an image optically captured by the second camera 52, to identify medical instruments 10 depicted in the image, to generate corresponding identity data, to determine the arrangement of the identified medical instruments 10 relative to the instrument carrier 20 and to store it as a target arrangement.

The computer 70 is further provided and configured to receive an image signal representing an image optically captured by the third camera 62, to identify medical instruments 10 depicted in the image, to generate corresponding identity data, to determine the arrangement of the identified medical instruments 10 relative to the instrument carrier 20, to compare the identities of the identified medical instruments with a target equipment, to compare the determined arrangement of the identified medical instruments 10 with a target arrangement and to generate a notification signal depending on these comparisons.

For this purpose, the computer 70 has in particular corresponding software which in particular controls the method steps described with reference to FIGS. 4 to 6.

Deviating from the representation in FIG. 1, only two regions or only one region can be provided instead of three regions 40, 50, 60. Accordingly, only two cameras or only one camera and only two or only one RFID writer and reader can be provided. In that case, the two regions or one region is large enough to accommodate a medical instrument 10 and the instrument carrier 20 side by side, or the instrument carrier 20 and the sterile container 30 side by side, or the medical instrument 10, the instrument carrier 20 and the sterile container 30 side by side. The system can be provided and configured only for optically capturing the medical instrument 10 and the instrument carrier 20 or only for optically capturing the instrument carrier 20 and the sterile container 30 or for optically capturing the medical instrument 10 and the instrument carrier 20 and the sterile container 30. In the same region, a medical instrument 10 and an instrument carrier 20 or an instrument carrier 20 and a sterile container 30 or a medical instrument 10, an instrument carrier 20 and a sterile container 30 can be optically captured one after the other by a camera. In the same region, RFID tags of a medical instrument 10 and an instrument carrier 20 or RFID tags of an instrument carrier 20 and a sterile container 30 or RFID tags of a medical instrument 10, an instrument carrier 20 and a sterile container 30 can be read and/or written to one after the other by a single RFID writer and reader.

Also, deviating from the representation in FIG. 1, one or more devices for moving—in particular horizontally—the storage surface 18, the instrument carrier 20 and/or the sterile container 30 between the regions 40, 50, 60 can be provided. Deviating from the representation in FIG. 1, one or more devices for moving—in particular horizontally—the first camera 42, the RFID reader 44, the camera 52, the RFID writer 54, the first light source 56, the first projection device 58, the third camera 62, the RFID writer and reader 64, the second light source 66 and/or the third light source 68 can be provided in order to break up the rigid assignment of the regions 40, 50, 60 to their functions described above.

FIG. 2 shows a schematic and enlarged representation of a data device, namely of the RFID tag 24 or the—in particular identical—RFID tag 34, each without the instrument carrier or the sterile container. The representation shows a power supply device 82, a microcontroller 84, a data memory 86 and a transmitting and receiving device 88 in an indicated cuboid-shaped and transparent housing.

The power supply device 82 is connected, on the one hand, to the photoelectric component 26, 36 and draws electrical power from it. The power supply device 82 in particular generates a constant predetermined voltage level. For this purpose, the power supply device 82 can contain an energy storage that compensates for power peaks—for example due to pulsed light output—and load peaks. The power supply device 82 provides electrical power to the microcontroller 84 and, via the latter, also to the data memory 86 and the transmitting and receiving device 88. The power supply device 82 is connected to the microcontroller 84 for this purpose. Deviating from the representation in FIG. 2, the power supply device 82 can also be directly connected to the data memory 86 and/or the transmitting and receiving device 88 in order to directly provide electrical power to them.

The microcontroller 84 is connected to the data memory 86 and the transmitting and receiving device 88 via data lines. The microcontroller 84 transmits data to be stored or the addresses of data to be read to the data memory 86 and data to be sent to the transmitting and receiving device 88. The microcontroller 84 receives data read from the data memory 86 and data received from the transmitting and receiving device 88.

The data memory 86 is in particular a non-volatile data memory that retains stored data even without a power supply. The data memory 86 is provided and configured to store identity data. The data memory 86 of the RFID tag 24 permanently connected to the instrument carrier 20 is provided for storing the identity data of the medical instruments 10 on the instrument carrier 20 and optionally also the identity of the instrument carrier 20 itself. The data memory 86 of the RFID tag 34 permanently connected to the sterile container 30 is provided for storing the identity data of the medical instruments 10 on the instrument carrier(s) 20 in the sterile container 30 and optionally also the identity data of the instrument carrier(s) 20 and/or the sterile container 30 itself. When reading identity data from the data memory 86, all stored identity data or individually addressed identity data can be read.

The transmitting and receiving device 88 is schematically shown as a planar coil in FIG. 1. In addition to such an antenna, the transmitting and receiving device 88 can comprise amplifiers, filters, digital-analog converters and analog-digital converters, etc. They can alternatively be integrated into the microcontroller 84. The transmitting and receiving device 88 complies with an RFID standard and transmits and receives data inductively, capacitively or in the form of electromagnetic waves.

As long as the photoelectric component 26, 36 receives sufficient power, the RFID tag 24, 34 does not depend on drawing power from the magnetic, electric or electromagnetic alternating field. The RFID tag 24, 34 may be provided and configured to provide minimum functionality without power being supplied by the photoelectric component 26, 36. For example, the minimum functionality includes sending identity data comprising the identity of the RFID tag 24, 34 and the instrument carrier 20 or the sterile container 30 itself when a request signal of sufficient strength and energy content has been received.

FIG. 3 shows a schematic representation of a system for assisting in the handling of medical instruments 10, in particular during a medical procedure. Alternatively, the system shown in FIG. 3 can be used, for example, to assist in the handling of medical instruments 10 after cleaning, immediately before or after sterilization and before use, in particular during the equipment process. The system is similar to the system shown in FIG. 1. However, only the second region 50 with the second camera 52 and the first light source 56 is provided for providing light output for the photoelectric component 26 on an RFID tag 24 that is permanently mechanically connected to an instrument carrier 20. The first region 40 with the first camera 42 and the RFID reader 44 and the third region 60 with the third camera 62, the third RFID writer and reader 64, the second light source 66 and the third light source 68 are not provided.

The system shown in FIG. 3 is, in particular, provided and configured to assist in the handling of medical instruments 10 during a medical procedure. For this purpose, an instrument carrier 20 is arranged in the region 50, on which one or more medical instruments 10 are kept ready for use during the medical procedure. The camera 52 is provided and arranged for optically capturing the medical instruments 10 arranged on the instrument carrier 20. The RFID reader 54 is provided and configured to read identity data from the memory of the RFID tag 24 permanently connected to the instrument carrier 20. The computer 70 is provided and configured to evaluate an image signal from the camera 52, to identify medical instruments 10 depicted in the image captured by the camera 52 and to generate corresponding identity data, to compare the generated identity data with identity data read from the memory of the RFID tag 24 and to change an attribute in identity data for a medical instrument depending on the comparison. For this purpose, the computer 70 has in particular corresponding software which in particular controls the method steps described with reference to FIG. 7.

FIG. 4 shows a schematic flowchart of a method for assisting in the handling of medical instruments. The method can be carried out with the system shown in FIG. 1 or with the system shown in FIG. 3 and in particular also with the RFID tag 24, 34 shown in FIG. 2. However, the method can also be carried out with a system and with an instrument carrier which have features, properties and functions that differ from those shown in FIGS. 1, 2 and 3. Therefore, the following use of reference signs from FIGS. 1, 2, 3 is merely illustrative.

In a first step 101, an image of one or more medical instruments 10 on an instrument carrier 20 is optically captured by a camera 52. The camera 52 captures the image in particular in the infrared spectral range and/or in the spectral range visible to the healthy human eye and/or in the ultraviolet spectral range. The camera 42, 52 can capture the image monochromatically or simultaneously in several color channels. The camera 42, 52 can be monocular or a stereo camera.

In a subsequent step 102, an analog or digital image signal representing the captured image is generated. This happens, for example, in one or more image sensors of the camera 52.

In a subsequent step 103, the image signal is transmitted to an image signal evaluation device 70. Transmission takes place in particular via electrical or optical cables or via WLAN, Bluetooth, ZigBee or another radio connection. Transmission can be omitted insofar as the image evaluation device 70 is integrated into the camera 52.

In a subsequent step 104, the image signal is evaluated by the image signal evaluation device 70 in order to determine the identity of the instrument carrier 20, the identities and the arrangement of the medical instruments 10 depicted in the captured image relative to the instrument carrier 20. The arrangement of the medical instruments also includes, in particular, their orientation.

To determine the identity of the instrument carrier 20, an optically readable code on the instrument carrier 20 may optionally be read. Alternatively or additionally, the identity of the instrument carrier 20 can be read from an RFID tag 24 that is permanently mechanically connected to the instrument carrier 20. Alternatively or additionally, the identity of the instrument carrier 20 can be entered manually at an input device 72.

The identity of each depicted medical instrument 10 is determined, for example, based on an optically readable code 12 on the medical instrument 10 and/or based on optically recognizable features, such as size, shape and/or colors. For this purpose, a database is used in which corresponding features are stored and assigned to individual types of medical instruments or to individual medical instruments. If a medical instrument 10 has an RFID tag, the RFID tag can alternatively or additionally be read by an RFID reader 44 to determine the identity of the medical instrument 10.

The image signal evaluation device 70 generates identity data representing the identities of the depicted medical instruments 10 and arrangement data representing the arrangement of the depicted medical instruments 10 relative to the instrument carrier 20.

The image signal evaluation device 70 is in particular a computer with software for image evaluation.

In a subsequent step 105, the identity data and the arrangement data are transmitted to a data memory 76, 86. The data memory can be integrated into the image evaluation device 70, for example as a mass memory. Alternatively, the data memory can be provided as a device 76 separate from the image evaluation device 70 or in a device separate from the image evaluation device 70. For example, the data memory 76 is part of a database in a server of a hospital or other medical facility. In that case, transmission takes place in particular via electrical or optical cables or via WLAN, Bluetooth, ZigBee or another radio connection.

Alternatively, the identity data and the arrangement data are transmitted to a data memory 86 in an RFID tag 20 that is permanently mechanically connected to the instrument carrier 20. In that case, transmission takes place in particular in accordance with an RFID standard. Furthermore, in that case, determination of the identity of the instrument carrier 20 in step 104 can be omitted.

In a subsequent step 106, the identity data and the arrangement data are stored in the data memory 76, 86.

The stored identity data and arrangement data represent the target equipment of the instrument carrier 20 and the target arrangement of the medical instruments 10 on the instrument carrier 20. During each subsequent equipment of the instrument carrier 20, the stored identity data and arrangement data can be used to control the equipment of the instrument carrier 20 by a person or a device. During each subsequent equipment of the instrument carrier 20, the actually generated equipment and arrangement of the medical instruments 10 on the instrument carrier 20 can be compared with the stored identity data and the stored arrangement data in order to check whether the instrument carrier 20 is correctly equipped.

FIG. 5 shows a schematic flowchart of another method for assisting in the handling of medical instruments. The method can be carried out with the system shown in FIG. 1 and in particular also with the RFID tag 24, 34 shown in FIG. 2. However, the method can also be carried out with a system and with an instrument carrier which have features, properties and functions that differ from those shown in FIGS. 1, 2. Therefore, the following use of reference signs from FIGS. 1, 2 is merely illustrative.

Several steps of the method shown in FIG. 5 are similar to steps of the method illustrated with reference to FIG. 4 and may have the features and properties described with reference to FIG. 4 and may be carried out in the manner described with reference to FIG. 4. The same applies to the devices required or used and their features, properties and functions, which may be similar or equivalent to those shown in FIG. 4.

In a first step 111, an image of a medical instrument 10 is optically captured by a camera 42. The medical instrument 10 lies in particular on a storage surface 18. Alternatively, the medical instrument 10 can be manually held in the field of view of the camera 42. Alternatively, the medical instrument 10 can lie on an instrument carrier 20 while the camera 42 captures the image.

In a subsequent step 112, an analog or digital image signal representing the captured image is generated.

In a subsequent step 113, the image signal is transmitted to an image signal evaluation device 70.

In a subsequent step 114, the image signal is evaluated by the image signal evaluation device 70 in order to determine the identity of the medical instrument 10 depicted in the captured image. The image signal evaluation device generates identity data that represent the identity of the depicted medical instrument 10. If a medical instrument 10 has an RFID tag, the RFID tag can alternatively or additionally be read by an RFID reader 44 to determine the identity of the medical instrument 10.

In a subsequent step 116, the target arrangement on an instrument carrier 20 assigned to the identified medical instrument 10 is determined. In that case, arrangement data representing the target arrangement of the identified medical instrument are read from a data memory 76, 86. The same as described in FIG. 4 applies to the data memory. In particular, it is the same data memory 76, 86 in which the target arrangement was previously stored with the steps shown in FIG. 4.

In a subsequent step 118, the determined target arrangement of the medical instrument 10 represented by the arrangement data is displayed. In particular, a projection device 58 projects the target arrangement onto the instrument carrier 20. Alternatively, the target arrangement can be displayed, for example, by a screen or other output device 74.

The method steps illustrated in FIG. 5 can simplify the equipment process of an instrument carrier 20 and thereby also reduce the error rate.

Some of the steps illustrated in FIG. 5 are optional. For example, steps 111, 112, 113, 114 may be omitted if the medical instrument 10 has an RFID tag. In that case, the RFID tag of the medical instrument 10 is read instead to determine its identity. Alternatively, steps 111, 112, 113, 114 are carried out and the RFID tag of the medical instrument 10 is read in order to obtain increased security in determining the identity of the medical instrument 10 through this redundancy.

FIG. 6 shows a schematic flowchart of another method for assisting in the handling of medical instruments. The method can be carried out with the system shown in FIG. 1, with the system shown in FIG. 3 and in particular also with the RFID tag 24, 34 shown in FIG. 2. However, the method can also be carried out with a system and with an instrument carrier which have features, properties and functions that differ from those shown in FIGS. 1, 2 and 3. Therefore, the following use of reference signs from FIGS. 1, 2, 3 is merely illustrative.

Several steps of the method shown in FIG. 6 are similar to steps of the methods illustrated with reference to FIGS. 4, 5 and may have the features and properties described with reference to FIGS. 4, 5 and may be carried out in the manner described with reference to FIGS. 4, 5. The same applies to the devices required or used and their features, properties and functions, which may be similar or equivalent to those shown in FIGS. 4, 5.

In a first step 121, an image of one or more medical instruments 10 on an instrument carrier is optically captured by a camera 52.

In a subsequent step 122, an image signal representing the captured image is generated.

In a subsequent step 123, the image signal is transmitted to an image signal evaluation device 70.

In a subsequent step 124, the image signal is evaluated by the image signal evaluation device 70 in order to determine the identity of the instrument carrier 20, the identities and the arrangement of the medical instruments 10 depicted in the captured image relative to the instrument carrier 20. The arrangement of the medical instruments 10 also includes, in particular, their orientation.

The determination of the identity of the instrument carrier 20 and the determination of the identities and arrangement of the medical instruments 10 are carried out in particular as shown in FIG. 4 in the context of step 104.

When determining the identities and arrangement of the medical instruments 10, equipment data are generated. The equipment data include identity data representing the identities of the depicted medical instruments 10 and optionally also arrangement data representing the arrangement of the depicted medical instruments 10 relative to the instrument carrier 20.

In a subsequent step 126, target equipment data assigned to the identified instrument carrier 20 are read from a data memory 76, 86, in particular by the image evaluation device 70. The target equipment data represent the target equipment of the identified instrument carrier 20, i.e., they comprise in particular the identity data of all medical instruments that are arranged on the instrument carrier during the target equipment. The target equipment data optionally also include target arrangement data which represent the target arrangement of the medical instruments on the instrument carrier 20. The same applies to the data memory 76, 86 as described in FIG. 4. It is, in particular, the same data memory 76, 86.

In a subsequent step 127, the determined identity data are compared with the read target equipment and the determined arrangement is compared with the read target arrangement, in particular by the image evaluation device 70.

In a subsequent step 128, a notification signal is generated depending on the comparison in step 127 which indicates whether the determined equipment corresponds to the read target equipment of the instrument carrier 20 and whether the determined arrangement of the medical instrument(s) 10 on the instrument carrier 20 corresponds to the read target arrangement. If the determined equipment does not correspond to the read target equipment or if the determined arrangement of the medical instrument(s) 10 on the instrument carrier 20 does not correspond to the read target arrangement, the notification signal can further comprise information about the deviation and/or instructions for a correction. In case of correct equipment and arrangement, the notification signal can trigger or control the transportation of the instrument carrier 20 to a next treatment station.

The notification signal can be generated by the output device 74 and can be immediately perceivable by a person acoustically, visually or tactilely. Alternatively, the notification signal may be an analog or digital signal transmitted to an output device 74 or to another device. The notification signal can be transmitted in particular to an industrial robot or multi-axis motion machine or a controller for the same. In case of incorrect equipment and arrangement, the notification signal can trigger or control a correction of the equipment or arrangement by the industrial robot.

In a subsequent step 135, the identity data and optionally also the arrangement data of the medical instruments 10 arranged on the instrument carrier 20 are transmitted to an RFID tag 24 that is permanently mechanically connected to the instrument carrier 20. This is done in particular by an RFID writer 54 and in accordance with an RFID standard.

In a subsequent step 136, the transmitted identity data and optionally also the arrangement data of the medical instruments 10 arranged on the instrument carrier 20 are stored in a data memory 86 of the RFID tag 24 that is permanently mechanically connected to the instrument carrier 20.

The method steps illustrated in FIG. 6 allow for checking the equipment of an instrument carrier 20, including the arrangement of the medical instruments 10 thereon, for example for quality assurance purposes. For this purpose, the method steps illustrated in FIG. 6 are carried out in particular according to the method steps illustrated in FIG. 5. Alternatively, the method steps illustrated in FIG. 6 can be carried out after the instrument carrier 20 has been mechanically equipped.

Some of the steps illustrated in FIG. 6 are optional. For example, steps 121, 122, 123, 124, 126, 127, 128 may be omitted if the equipment of the instrument carrier 20 is known with sufficient certainty and the probability of incorrect equipment is sufficiently low. Furthermore, for example, steps 135, 136 can be omitted and identity data can instead be stored in another data memory 76 and assigned there to the instrument carrier 20.

FIG. 7 shows a schematic flowchart of another method for assisting in the handling of medical instruments. The method can be carried out with the system shown in FIG. 1, with the system shown in FIG. 3 and in particular also with the RFID tag 24, 34 shown in FIG. 2. However, the method can also be carried out with a system and with an instrument carrier which have features, properties and functions that differ from those shown in FIGS. 1, 2 and 3. Therefore, the following use of reference signs from FIGS. 1, 2, 3 is merely illustrative.

Several steps of the method shown in FIG. 7 are similar to steps of the methods illustrated with reference to FIGS. 4, 5 and especially 6 and may have the features and properties described with reference to FIGS. 4, 5, 6 and may be carried out in the manner described with reference to FIGS. 4, 5, 6. The same applies to the devices required or used and their features, properties and functions, which may be similar or equivalent to those shown in FIGS. 4, 5, 6.

In a first step 140, an instrument carrier 20 is placed into a sterile container 30. Before the first step 140, the instrument carrier 20 may have been equipped with the steps shown in FIG. 5. Furthermore, the equipment of the instrument carrier 20 may have been checked before the first step using the steps shown in FIG. 6. If the sterile container 30 is provided to accommodate several instrument carriers 20 in a stack, the subsequent method steps shown in FIG. 7 relate in particular to the topmost instrument carrier 20 introduced last.

The following steps 141, 142, 143, 144, 146, 147, 148 correspond in particular largely to steps 121, 122, 123, 124, 126, 127, 128 shown in FIG. 6. Steps 141, 142, 143, 144, 146, 147, 148 differ from steps 121, 122, 123, 124, 126, 127, 128 shown in FIG. 6 in particular only in that the instrument carrier 20 is arranged in the sterile container. Steps 141, 142, 143, 144, 146, 147, 148 serve to check the correct equipment of the instrument carrier 20 and the correct arrangement of the medical instruments 10 in the instrument carrier 20.

In a subsequent step 154, the identity data and optionally also the arrangement data of the medical instruments 10 arranged on the instrument carrier 20 are read from the RFID tag 24 that is permanently mechanically connected to the instrument carrier 20. Transmission is carried out in particular by an RFID writer and reader 54 and in accordance with an RFID standard.

In a subsequent step 155, the identity data read in the preceding step and optionally also the arrangement data of the medical instruments 10 arranged on the instrument carrier 20 are transmitted to an RFID tag 34 that is permanently mechanically connected to the sterile container 30. Optionally, identity data representing the identity of the instrument carrier 20 are also transmitted to an RFID tag 34 that is permanently mechanically connected to the sterile container 30. Transmission is carried out in particular by the RFID writer and reader 54 and in accordance with an RFID standard.

In a subsequent step 156, the transmitted identity data and optionally also the arrangement data of the medical instruments 10 arranged on the instrument carrier 20 are stored in a data memory 86 of the RFID tag 34 that is permanently mechanically connected to the sterile container 30.

The method steps illustrated in FIG. 7 allow for checking the equipment of an instrument carrier 20, including the arrangement of the medical instruments 10 thereon, for example for quality assurance purposes. For this purpose, the method steps illustrated in FIG. 7 are carried out in particular according to the method steps illustrated in FIGS. 5 and 6. Alternatively, the method steps illustrated in FIG. 7 can be carried out after the instrument carrier 20 or sterile container 30 has been mechanically equipped.

Some of the steps illustrated in FIG. 7 are optional. For example, steps 141, 142, 143, 144, 146, 147, 148 may be omitted if the equipment of the instrument carrier 20 is known with sufficient certainty and the probability of incorrect equipment is sufficiently low.

Furthermore, step 154 may be omitted if the identity data and optionally also the arrangement data are stored in another memory 76. In that case, the identity data and optionally also the arrangement data can instead be read from the other memory, for example a central database 76.

Furthermore, steps 155, 156 may be omitted. Instead, the identity data and optionally also the arrangement data of the medical instruments 10 arranged on the instrument carrier 20 together with identity data representing the identity of the instrument carrier 20 can be stored in another memory, for example a central database 76.

FIG. 8 shows a schematic flowchart of another method for assisting in the handling of medical instruments. The method can be carried out with the system shown in FIG. 1, with the system shown in FIG. 3 and in particular also with the RFID tag 24, 34 shown in FIG. 2. However, the method can also be carried out with a system and with an instrument carrier which have features, properties and functions that differ from those shown in FIGS. 1, 2 and 3. Therefore, the following use of reference signs from FIGS. 1, 2, 3 is merely illustrative.

Several steps of the method shown in FIG. 8 are similar to steps of the methods illustrated with reference to FIGS. 4, 5, 6, 7 and may have the features and properties described with reference to FIGS. 4, 5, 6, 7 and may be carried out in the manner described with reference to FIGS. 4, 5, 6, 7. The same applies to the devices required or used and their features, properties and functions, which may be similar or equivalent to those shown in FIGS. 4, 5, 6, 7.

In a first step 160, an instrument carrier 20 is provided for use during a medical procedure. The equipment of the instrument carrier 20, i.e., the identities of the medical instruments 10 arranged thereon and optionally also their arrangement and orientation are adapted to the medical procedure. This means that an instrument carrier 20 with suitable equipment is selected for the medical procedure.

The provided instrument carrier 20 and the medical instruments 10 arranged thereon are in particular sterile until they are provided. For this purpose, they were in particular autoclaved. During the medical procedure, medical instruments 10 are taken from the instrument carrier 20 and used. They are then no longer sterile.

During the medical procedure, steps 161, 162, 163, 164, which largely correspond to steps 121, 122, 123, 124 shown in FIG. 6 and steps 141, 142, 143, 144 shown in FIG. 7, are carried out. They are carried out in particular by and with the system shown in FIG. 3. In step 164, determining the arrangement of the medical instruments 10 depicted in the captured image is optional.

In a subsequent step 166, target equipment data assigned to the instrument carrier 20, i.e., the identity data of the medical instruments arranged on the instrument carrier 20 during the target equipment process, are read from a data memory 76, 86. The target equipment data optionally also include target arrangement data which represent the target arrangement of the medical instruments on the instrument carrier 20. The same as described in FIG. 4 applies to the data memory 76, 86. It is, in particular, the same data memory 76, 86.

In a subsequent step 167, the determined equipment data are compared with the target equipment data read from the memory 76, 86, for example by the image evaluation device 70.

In a subsequent step 168, the attribute “non-sterile” is set for a medical instrument 10 which, on the one hand, is part of the target equipment of the instrument carrier 20, but, on the other hand, is not part of the determined equipment. This attribute is stored in particular in the memory 86 of the RFID tag 24 permanently connected to the instrument carrier 20 and/or in a central database 76 or in another memory. The attribute “non-sterile” indicates that the medical instrument must be sterilized before any further use.

In step 168, a counter that counts the number of uses of the medical instrument may be incremented simultaneously. Comparing the counter with a predetermined upper limit of uses allows for the timely removal of the medical instrument from the inventory of a hospital, doctor's office or other medical facility.

During the medical procedure, steps 161, 162, 163, 164, 167, 168, and optionally also step 166, are carried out in particular repeatedly.

Below are described some of the potential variations of the embodiments disclosed herein.

A system for assisting in the handling of medical instruments (10), having: a camera (42, 52, 62) for optically capturing an image of one or more medical instruments (10) and for providing an image signal representing the captured image; an image evaluation device (70) for receiving the image signal, for identifying one or more medical instruments (10) depicted in the image represented by the image signal, and for providing identification data identifying the depicted instruments (10); a data transmission device (56, 66) for sending identification data to a data receiving device (88) of a data device (22) permanently connected to an autoclavable instrument carrier (20).

The system described above, further having: an autoclavable instrument carrier (20) for storing, transporting and keeping medical instruments ready (10); an autoclavable data device (22) permanently connected to the autoclavable instrument carrier (20) having a data receiving device (88) for receiving data and a repeatedly writable and readable memory (86) for storing data received by the data receiving device (88).

A system described above, further having: an autoclavable power receiving device (24) permanently connected to the autoclavable instrument carrier (20) for receiving power in the form of light and for providing electrical power to the data device (22).

A system described above, further having: a light source (56, 66) for providing power in the form of light for the power receiving device (24) permanently connected to the autoclavable instrument carrier.

A system described above, further having: a projection device (58) for visibly projecting an intended target position of a medical instrument (10) onto a surface of the autoclavable instrument carrier (20).

A system described above, further having: a database (76) in which intended positions of medical instruments (10) are stored on a surface of the autoclavable instrument carrier (20).

An autoclavable instrument carrier (20) for storing, transporting and keeping medical instruments (10) ready, having: an autoclavable data device (24) permanently connected to the autoclavable instrument carrier (20) having a repeatedly writable and readable memory (86) and a data transmission device (88) for receiving and sending data; an autoclavable energy receiving device (26) permanently connected to the autoclavable instrument carrier (20) for receiving power in the form of light and for providing electrical power to the data device (22).

The autoclavable instrument carrier (20) described above, further having: a barcode or other one- or two-dimensional optically readable code (22) or other optically readable device for identifying the autoclavable instrument carrier (20).

An autoclavable sterile container (30) for storing, transporting and keeping ready one or more instrument carriers (20) each with one or more medical instruments (10), having: an autoclavable data device (34) permanently connected to the autoclavable sterile container (30) having a repeatedly writable and readable memory (86) and a data transmission device (88) for sending and receiving data; an autoclavable energy receiving device (36) permanently connected to the autoclavable sterile container (30) for receiving power in the form of light and for providing electrical power to the data device (32).

The autoclavable sterile container (30) described above, further having: a barcode or other one- or two-dimensional optically readable code (32) or other optically readable device for identifying the autoclavable sterile container (30).

An autoclavable instrument carrier (20) described above, or an autoclavable sterile container (30) described above, wherein the energy receiving device (26, 36) comprises a photovoltaic cell or an optoelectric converter based on gallium arsenide.

A method for assisting in the handling of medical instruments (10), comprising the steps of: optically capturing (101; 111; 121; 141) an image of one or more medical instruments (10); generating (102; 112; 122; 142) an image signal representing the captured image; transmitting (103; 113; 123; 143) the image signal to an image evaluation device (70); evaluating (104; 114; 124; 144) the image signal with the image evaluation device (70) in order to identify the medical instrument(s) (10) depicted in the image and generating identity data representing the identities of the identified medical instrument(s) (10); transmitting (135) identity data; storing (136) the generated identity data in a memory (86) permanently connected to an instrument carrier (20) or comparing (125) the generated identity data with identity data stored in the memory (86) permanently connected to the instrument carrier (20).

The method described above, wherein evaluating (104) the image signal with the image evaluation device (70) comprises capturing the arrangement of the medical instruments (10) depicted in the image, further comprising the step of: storing (106) the captured arrangement as the target arrangement of the medical instruments (10).

A method described above, further comprising the steps of: determining (116) a target arrangement of an identified medical instrument (10) on an instrument carrier (20); projecting (117) the medical instrument (10) in the target arrangement onto the instrument carrier (20).

A method described above, further comprising the steps of: optically capturing (121; 141) another image of the medical instrument(s) (10) on the instrument carrier (20); generating (122; 142) another image signal representing the captured additional image; transmitting (123; 143) the further image signal to the image evaluation device (70); evaluating (124; 144) the further image signal with the image evaluation device (70) in order to determine the arrangement of the medical instrument(s) (10) depicted in the further image; generating (128; 148) a notification signal depending on whether the specific arrangement of the medical instrument(s) (10) corresponds to their target arrangement.

A method described above, wherein generating (128; 146) the notification signal is further dependent on whether storing (136; 156) the identity data of the identified medical instrument(s) (10) in the memory (86) permanently connected to the instrument carrier (20) was successful.

A method described above, further comprising the step of: setting (167) the attribute “non-sterile” to a medical instrument (10) if the comparison of the identity data shows that the medical instrument (10) is no longer arranged on the instrument carrier (20).

A method described above, further comprising the steps of: introducing (140) the instrument carrier (20) into a sterile container (30); reading (154) the identity data of the identified medical instrument(s) (10) from the memory (86) permanently connected to the instrument carrier (20); transmitting (155) the identity data of the identified medical instrument(s) (10) to a memory (86) permanently connected to the sterile container (30); storing (156) the identity data of the identified medical instrument(s) (10) in the memory (86) permanently connected to the sterile container (30).

### REFERENCE SIGNS

10 medical instrument
12 optically readable code on the medical instrument 10
18 storage surface for the medical instrument 10
20 autoclavable instrument carrier (in particular sieve, tray, etc.)
22 optically readable code on the autoclavable instrument carrier 20
24 RFID tag on the autoclavable instrument carrier 20
26 photoelectric component on the RFID tag 24 on the autoclavable instrument carrier 20 for receiving optical power and for providing electrical power for the RFID tag 24
28 outline of the medical instrument 10 projected onto the autoclavable instrument carrier 20 in a target position
30 autoclavable sterile container
32 optically readable code on the autoclavable sterile container 30
34 RFID tag on the autoclavable sterile container 30
36 photoelectric component on the RFID tag 34 on the autoclavable sterile container 30 for receiving optical power and for providing electrical power for the RFID tag 34
40 first region for identifying a medical instrument 10
42 first camera, for optically capturing a medical instrument 10 on the storage surface 18
44 RFID reader for the first region 40
50 second region for equipping an instrument carrier 10
52 second camera, for capturing a medical instrument 10 on the autoclavable instrument carrier 20
54 RFID writer for the second region 50
56 first light source for providing light output for the photoelectric component 26 on the RFID tag 24
58 projection device for projecting the outline 28 of the medical instrument 10 in a target position and target orientation onto the autoclavable instrument carrier 20
60 third region for filling a sterile container 30
62 camera for capturing a medical instrument 10 on the autoclavable instrument carrier 20 in the sterile container 30
64 RFID writer and reader for the third region 60
66 second light source for providing light output for the photoelectric component 36 on the RFID tag 34 on the autoclavable instrument carrier 20
68 third light source for providing light output for the photoelectric component 36 on the RFID tag 34 on the autoclavable sterile container 30
70 computer as a device for image evaluation
72 input device as part of a user interface; in particular keyboard
74 output device as part of a user interface; in particular screen
76 database
82 power supply device of RFID tag 22, 32
84 microcontroller of RFID tag 22, 32
86 data memory of RFID tag 22, 32
88 transmitting and receiving device of RFID tag 22, 32
101 step (optically capturing an image of one or more medical instruments)
102 step (generating an image signal representing the captured image)
103 step (transmitting the image signal to an image evaluation device)
104 step (evaluating the image signal with the image evaluation device with regard to identity, arrangement of the medical instrument(s), and generating identity data and arrangement data)
105 step (transmitting the identity data and arrangement data)
106 step (saving the arrangement as a target arrangement)
111 step (optically capturing an image of one or more medical instruments)
112 step (generating an image signal representing the captured image)
113 step (transmitting the image signal to an image evaluation device)
114 step (evaluating the image signal with the image evaluation device 70 with regard to the identity of the medical instrument, and generating identity data)
116 step (determining the target arrangement of the one or more identified medical instruments 10)
118 step (projecting the medical instrument(s) 10 in the determined target arrangement onto the instrument carrier 20)
121 step (optically capturing another image of one or more medical instruments 10 on an instrument carrier 20)
122 step (generating another image signal representing the captured additional image)
123 step (transmitting the further image signal to an image evaluation device)
124 step (evaluating the further image signal with the image evaluation device 70 with regard to the identity, arrangement and orientation of the medical instrument(s), and generating identity data)
126 step (reading the target equipment of the identified instrument carrier 20 and the target arrangement of the medical instrument(s) 10 on the instrument carrier 20)
127 step (comparing the determined equipment with the read target equipment and the determined arrangements with the read target arrangement)
128 step (step (generating a notification signal)
135 step (transmitting the identity data to a data memory 86 permanently connected to the instrument carrier 20)
136 step (storing the identity data in the memory 86 permanently connected to the instrument carrier 20)
140 step (introducing the instrument carrier 20 into a sterile container 30)
141 step (optically capturing another image of one or more medical instruments 10 on the instrument carrier 20 in the sterile container 30)
142 step (generating another image signal representing the captured additional image)
143 step (transmitting the further image signal to an image evaluation device)
144 step (evaluating the further image signal with the image evaluation device 70 with regard to the identity, arrangement and orientation of the medical instrument(s), and generating identity data)
146 step (reading the target equipment of the identified instrument carrier 20 and the target arrangement of the medical instrument(s) 10 on the instrument carrier 20)
147 step (comparing the determined equipment with the read target equipment and the determined arrangements with the read target arrangement)
148 step (step (generating a notification signal)
154 step (reading the identity data from the data device 24 permanently connected to the instrument carrier 20
155 step (transmitting the identity data to a data memory 86 permanently connected to a sterile container 30)
156 step (storing the identity data in the memory 86 permanently connected to the sterile container 30)
160 step (providing the instrument carrier 20 for a medical procedure)
161 step (optically capturing another image of one or more medical instruments 10 on the instrument carrier 20)
162 step (generating another image signal representing the captured additional image)
163 step (transmitting the further image signal to an image evaluation device)
164 step (evaluating the further image signal with the image evaluation device 70 with regard to the identity and o, arrangement and orientation of the medical instrument(s), and generating identity data)
166 step (reading the target equipment from a memory 86 permanently connected to the instrument carrier 20)
167 step (comparing the determined equipment with the read target equipment)
168 step (setting the attribute “non-sterile” to a medical instrument 10 if the comparison of the identity data shows that the medical instrument 10 is no longer arranged on the instrument carrier 20)
168 step (increasing a counter that counts the uses of the medical instrument by one)

1. A system for assisting in the handling of medical instruments, comprising:
a camera for optically capturing an image of one or more medical instruments and for providing an image signal representing the captured image;
an image evaluation device for receiving the image signal, for identifying one or more medical instruments depicted in the image represented by the image signal, and for providing identification data identifying the depicted instruments;
a data transmission device for sending identification data to a data receiving device of a data device permanently connected to an autoclavable instrument carrier.

2. The system of claim 1, further comprising:
an autoclavable instrument carrier for storing, transporting and keeping medical instruments ready;
an autoclavable data device permanently connected to the autoclavable instrument carrier having a data receiving device for receiving data and a repeatedly writable and readable memory for storing data received by the data receiving device.

3. The system of claim 2, further comprising:
an autoclavable power receiving device permanently connected to the autoclavable instrument carrier for receiving power in the form of light and for providing electrical power to the data device.

4. The system of claim 3, further comprising:
a light source for providing power in the form of light for the power receiving device permanently connected to the autoclavable instrument carrier.

5. The system of claim 1, further comprising:
a projection device for visibly projecting an intended target position of a medical instrument onto a surface of the autoclavable instrument carrier.

6. The system of claim 2, further comprising:
a projection device for visibly projecting an intended target position of a medical instrument onto a surface of the autoclavable instrument carrier.

7. The system of claim 3, further comprising:
a projection device (58) for visibly projecting an intended target position of a medical instrument (10) onto a surface of the autoclavable instrument carrier (20).

8. The system of claim 4, further comprising:
a projection device for visibly projecting an intended target position of a medical instrument onto a surface of the autoclavable instrument carrier.

9. The system of claim 1, further comprising:
a database in which intended positions of medical instruments are stored on a surface of the autoclavable instrument carrier.

10. The system of claim 8, further comprising:
a database in which intended positions of medical instruments are stored on a surface of the autoclavable instrument carrier.

11. A method for assisting in the handling of medical instruments, comprising the steps of:
optically capturing an image of one or more medical instruments;
generating an image signal representing the captured image;
transmitting the image signal to an image evaluation device;
evaluating the image signal with the image evaluation device in order to identify the medical instrument or instruments depicted in the image and generating identity data representing the identities of the identified medical instrument or instruments;
transmitting identity data;
storing the generated identity data in a memory permanently connected to an instrument carrier or comparing the generated identity data with identity data stored in the memory permanently connected to the instrument carrier.

12. The method of claim 11, wherein the step of evaluating the image signal with the image evaluation device comprises capturing the arrangement of the medical instruments depicted in the image, and
the method further comprises the step of:
storing the captured arrangement as the target arrangement of the medical instruments.

13. The method according to claim 11, further comprising the steps of:
determining a target arrangement of an identified medical instrument on an instrument carrier;
projecting the medical instrument in the target arrangement onto the instrument carrier.

14. The method of claim 11, further comprising the steps of:
optically capturing another image of the medical instrument or instruments on the instrument carrier;
generating another image signal representing the captured additional image;
transmitting the further image signal to the image evaluation device;
evaluating the further image signal with the image evaluation device in order to determine the arrangement of the medical instrument or instruments depicted in the further image;
generating a notification signal depending on whether the specific arrangement of the medical instrument or instruments corresponds to their target arrangement.

15. The method of claim 13, further comprising the steps of:
optically capturing another image of the medical instrument or instruments on the instrument carrier;
generating another image signal representing the captured additional image;
transmitting the further image signal to the image evaluation device;
evaluating the further image signal with the image evaluation device in order to determine the arrangement of the medical instrument or instruments depicted in the further image;
generating a notification signal depending on whether the specific arrangement of the medical instrument or instruments corresponds to their target arrangement.

16. The method of claim 14, wherein
generating the notification signal is further dependent on whether storing the identity data of the identified medical instrument or instruments in the memory permanently connected to the instrument carrier was successful.

17. The method of claim 16, further comprising the step of:
setting the attribute “non-sterile” to a medical instrument if the comparison of the identity data shows that the medical instrument is no longer arranged on the instrument carrier.

18. The method of claim 11, further comprising the steps of:
introducing the instrument carrier into a sterile container;
reading the identity data of the identified medical instrument or instruments from the memory permanently connected to the instrument carrier;
transmitting the identity data of the identified medical instrument or instruments to a memory permanently connected to the sterile container;
storing the identity data of the identified medical instrument or instruments in the memory permanently connected to the sterile container.

19. The method of claim 15, further comprising the steps of:
introducing the instrument carrier into a sterile container;
reading the identity data of the identified medical instrument or instruments from the memory permanently connected to the instrument carrier;
transmitting the identity data of the identified medical instrument or instruments to a memory permanently connected to the sterile container;
storing the identity data of the identified medical instrument or instruments in the memory permanently connected to the sterile container.

20. The method of claim 17, further comprising the steps of:
introducing the instrument carrier into a sterile container;
reading the identity data of the identified medical instrument or instruments from the memory permanently connected to the instrument carrier;
transmitting the identity data of the identified medical instrument or instruments to a memory permanently connected to the sterile container;
storing the identity data of the identified medical instrument or instruments in the memory permanently connected to the sterile container.

21. (canceled)

22. (canceled)

23. (canceled)

24. (canceled)

25. (canceled)

26. (canceled)

27. (canceled)

28. (canceled)
